(19) United States
(12) Patent Application Publication
(10) Pub. No.: US 2017/0014048 A1
(43) Pub. Date: Jan. 19, 2017

(54) RESPIRATORY SAMPLING WITH SUPPORT STRUCTURE

(71) Applicant: ORIDION MEDICAL 1987 LTD., Jerusalem (IL)

(72) Inventor: Thomas A. Wilke, Boulder, CO (US)

(21) Appl. No.: 14/798,481

(22) Filed: Jul. 14, 2015

(51) Int. Cl. A61B 5/097; A61B 5/08; A61B 5/00; A61M 16/08; A61M 16/06

(52) U.S. Cl. CPC A61B 5/097; A61M 16/085; A61M 16/0672; A61B 5/6803; A61B 5/082; A61B 5/7285

(57) ABSTRACT

The present disclosure provides an apparatus for collecting respiratory samples with a support structure designed to be wearable by a subject. The apparatus includes a sample collector and a tubing system for collecting and delivering the sample. Optionally, the sample collector and/or the tubing system is movable between a first and a second position to gain access to the mouth and/or nose of the patient. Wearable support device for and method of breath sampling are disclosed.

### TECHNICAL FIELD

The present disclosure generally relates to the field of breath sampling.

### BACKGROUND

Monitoring respiratory parameters of a subject requires obtaining breath samples from the mouth and/or nose thereof by a sample collector, and delivering the samples by a tubing system to a detector for detecting changes in concentrations of molecules of interest within the respiratory gasses. The sample collector and the tubing system for delivering the samples are mounted to the face of the subject and are generally inconvenient and uncomfortable to the subject as they touch his/her face and, in some cases, the tubing system is even attached to the face of the subject by tapes to affix them in a desired position. Additionally, gaining access to the mouth and/or nose of the patient is complicated and requires removing the sample collector and the tubing system and then remounting them back to the desired position by a professional care giver.

There is thus a need in the art for breath sampling devices that provide comfort to the subject under respiratory monitoring, and avail ease of access to the mouth and/or nose of the subject.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided herein devices, systems and methods for attaching a sample collector and cannula tubing system to a subject using a support structure. According to some embodiments, the support structure is configured to restrict the tubing system in certain positions. According to some embodiments, the support structure is configured to be wearable by the subject.

Advantageously, the device may provide convenience for the subject under monitoring as the tubing system is mounted on a support structure instead of being directly mounted to the face or head of the subject.

According to some embodiments, the support structure is configured to enable positioning the tubing system and/or the sample collector in at least two positions, a first “engaged” position and a second “retracted” position. In the engaged position, respiratory sample collection is enabled, while in the retracted position the sample collector and/or tubing system is retracted from the mouth and/or nose of the subject.

Advantageously, the device may provide convenient access to the mouth and/or nose of the patient by positioning the tubing system and/or sample collector in a retracted position, and resuming respiratory monitoring by positioning the tubing system and/or sample collectors in an engaged position.

According to some embodiments, there is provided a breath sampling apparatus, including a breath sampling tube, a connector connectable to or formed with the breath sampling tube configured to attach the breath sampling tube to a wearable support structure, and an oral/nasal breath sample collector configured to collect breath samples from a patient's respiratory opening and deliver the collected breath samples to the breath sampling tube, wherein the connector includes a positioning element movable between a first position and a second position such that at the first position the oral/nasal breath sample collector is approximated to the respiratory opening of the subject for collecting breath samples, and at the second position the oral/nasal breath sample collector is retracted from the respiratory opening of the subject to facilitate accessibility to the respiratory opening.

According to some embodiments, the wearable support structure includes eyeglasses.

According to some embodiments, the wearable support structure includes a head band, earphones, a headset, a hearing aid, a hair pin or a hair accessory.

According to some embodiments, the oral/nasal breath sample collector is configured to obtain respiratory samples from a mouth and/or one or two nostrils of the subject.

According to some embodiments, the oral/nasal breath sample collector further includes one or more oxygen delivery outlets.

According to some embodiments, the apparatus further includes an indication mechanism configured to provide indication related to a position of the breath sample collector to a breath analyzer, wherein the breath analyzer is configured to modulate breath sampling and/or breath analyzing upon indication that the positioning element is at the second position.

According to some embodiments, the apparatus further includes an indication mechanism configured to provide indication related to a position of the oral/nasal breath sample collector to a breath analyzer, wherein the breath analyzer is configured to activate breath sampling and/or breath analyzing upon indication that the positioning element is at the first position.

According to some embodiments, the apparatus further includes an indication mechanism configured to provide indication related to a position of the breath sample collector to a pump, wherein the pump is configured to stop breath sampling upon indication that the positioning element is at the second position.

According to some embodiments, the apparatus further includes an indication mechanism configured to provide indication related to a position of the breath sample collector to a pump, wherein the pump is configured to activate breath sampling upon indication that the positioning element is at the first position.

According to some embodiments, the indication mechanism is configured for manual actuation.

According to some embodiments, the indication mechanism is configured for automatic actuation upon changes in a position of the positioning element.

According to some embodiments, the breath sampling tube is pliable and configured to retain desired shapes.

According to some embodiments, the breath sampling tube includes a pliable element configured to retain desired shapes.

According to some embodiments, there is provided a wearable support device for breath sampling, the device including a support structure, wearable by a subject, and a connector connectable to or formed with the support structure and configured to attach the support structure to a breath sampling tube, wherein the connector includes a positioning element movable between a first position and a second position such that at the first position the breath sampling tube is approximated to the respiratory opening of the subject for collecting breath samples, and at the second position the breath sampling tube is retracted from the respiratory opening of the subject to facilitate accessibility to the respiratory opening.

According to some embodiments, the device further includes an indication mechanism configured to provide indication related to a position of the breath sampling tube to a breath analyzer, wherein the breath analyzer is configured to modulate breath sampling and/or breath analyzing upon indication that the positioning element is at the second position.

According to some embodiments, the indication mechanism is configured for manual actuation.

According to some embodiments, the indication mechanism is configured for automatic actuation upon changes in a position of the positioning element.

According to some embodiments, the frame includes an eyeglass frame.

According to some embodiments, the frame includes a headband.

According to some embodiments, there is provided a method for breath sampling from a subject, the method including connecting a breath sampling tube having an oral/nasal breath sample collector to a wearable support structure, such that the breath sampling tube is movable relative to the wearable support structure, using an indication mechanism, providing a signal indicative of a position of the oral/nasal breath sample collector to a breath analyzer and/or a pump, using processing circuitry, modulating the activity of the breath analyzer and/or the pump based on an indication received from the indication mechanism.

According to some embodiments, the breath sampling tube is flexible and configured to retain desired shapes by bending.

According to some embodiments, the method further includes adjusting the breath sampling tube to the subject by bending it.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a common placement of an ordinary breath sampling device on a subject;

FIG. 2A schematically illustrates a breath sampling device with a connector, according to some embodiments;

FIG. 2B schematically illustrates a breath sampling device with a connector and an eyewear support structure, according to some embodiments;

FIG. 3A schematically illustrates a breath sampling device with a connector, a positioning element and an eyewear support structure in a perspective view, according to some embodiments;

FIG. 3B schematically illustrates a breath sampling device with a connector, a positioning element and a headband support structure in a perspective view, according to some embodiments;

FIG. 4A schematically illustrates a breath sampling device with an eyewear support structure on a subject in an engaged position, according to some embodiments;

FIG. 4B schematically illustrates a breath sampling device with an eyewear support structure on a subject in a partially retracted position, according to some embodiments;

FIG. 5A schematically illustrates a breath sampling device with an eyewear support structure and a pliable element on a subject in an engaged position, according to some embodiments;

FIG. 5B schematically illustrates a breath sampling device with an eyewear support structure and a pliable element on a subject in a retracted position, according to some embodiments;

FIG. 6 schematically illustrates a breath sampling device with an eyewear support structure and an indication switch on a subject, according to some embodiments;

FIG. 7A schematically illustrates a breath sampling device with an eyewear support structure and a tube holder on a subject in an engaged position, according to some embodiments;

FIG. 7B schematically illustrates a breath sampling device with an eyewear support structure and a tube holder on a subject in a retracted position, according to some embodiments;

FIG. 8 illustrates a block diagram of a method for using a breath sampling device with a displaceable sample collector, according to some embodiments; and

FIG. 9 illustrates a block diagram of a method for using a breath sampling device with a displaceable sample collector and an indication mechanism, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Monitoring respiratory related parameters enables objective evaluation of the ventilatory/respiratory status of a patient and indirectly enables evaluation of the circulatory and metabolic status, among other conditions. The monitoring is generally done by obtaining respiratory samples from a subject, which requires a sample collector, and a tubing system for delivering the collected samples to a detector and/or analyzing unit.

Commonly, the tubing system and the sample collector are attached to the face of the subject at a desired position by a professional care giver and they cause inconvenience and limit the facial mobility of the subject, in addition to obstructing access to the mouth and/or nose of the subject for conducting regular activities such as oral or nasal administration of medications or simply drinking liquids and eating. To gain access to the mouth and/or nose of the subject, the professional care giver needs to remove the sample collector and the tubing system from the face of the subject, and to reattach them in the desired position once access to the mouth and/or nose is no longer required.

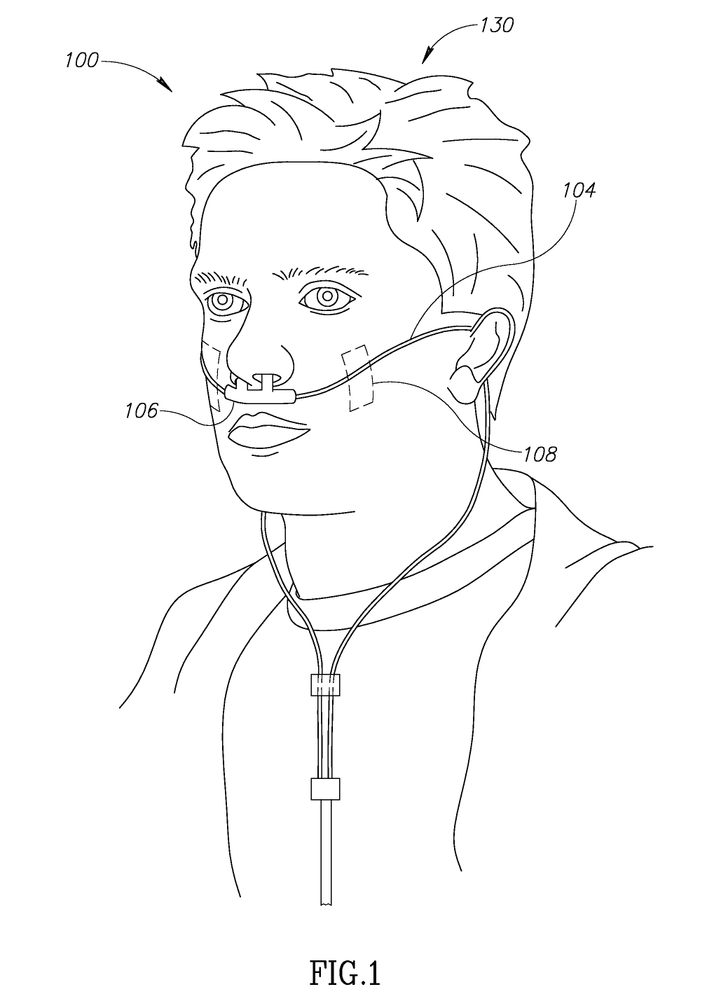

Reference is now made to FIG. 1, which schematically illustrates a common position of an ordinary breath sampling device 100 on a subject 130. Breath sampling device 100 includes a tubing system 104 configured to deliver breath samples from a sample collector 106 to be analyzed for evaluating respiratory related parameters. For sample collecting to be possible, sample collector 106 is positioned near, and sometimes partially inside, a respiratory opening of subject 130, and tubing system 104 is attached to the face of subject 130 using, for example, an adhesive tape 108 tubing system. Sample collector 106, tubing system 104 and adhesive tape 108 generally compromise the comfort of subject 100.

Conducting ordinary actions, such as administering nasal drops medications, wiping one's nose and, when sample collector 106 is an oral/nasal cannula rather than a nasal cannula, also eating, talking and other actions, require the removal of sample collector 106, tubing system 104 and adhesive tape 108 from the face of subject 130 and reattaching them once the ordinary action is complete in order to resume respiratory monitoring. The removal and reattachment of sample collector 106, tubing system 104 and adhesive tape 108 are done by a professional care giver and are time consuming and require effort.

Additionally, a medical care giver needs to pause or turn off the respiratory monitoring detector and/or the analyzing unit when sample collector 106 and tubing system 104 are removed, and to turn them back on when sample collector 106 and tubing system 104 are reattached.

A device for collecting respiratory samples that is easily repositioned by the subject/patient/care-provider without compromising the comfort and accessibility of the patient is thus desirable.

According to some embodiments, there is provided a device having a support structure configured to be wearable on the subject, wherein the tubing system for delivering the respiratory samples is connected to the support structure and a sample collector is connected to the tubing system.

Advantageously, the support structure holds the tubing system and the sample collector in a desired position for sampling respiratory gasses without adhesively attaching the tubing system to the face of the patient.

According to some embodiments, the tubing system and/or the sample collector are movable between two positions, a first “engaged” position, and a second “retracted” position.

According to some embodiments, the device is configured to enable respiratory sample collection in the engaged position, and to withdraw the sample collector and/or tubing system from the mouth and/or nose of the subject in the retracted position.

Advantageously, the device may provide convenient access to the mouth and/or nose of the patient by positioning the tubing system and/or sample collector in a retracted position, and resuming respiratory monitoring by positioning the tubing system and/or sample collectors in an engaged position.

According to some embodiments, the support structure may be an eyeglass frame. According to some embodiments, the device includes a button configured to be indicative of the position of the sample collector and/or tube.

According to some embodiments, the tubing system is made from or includes an elastic material, bendable to obtain a desired shape. According to some embodiments, the support structure holds the tubing system and the sample collector in an engaged position in a shape in which the tubing system does not come in contact with the face of the subject.

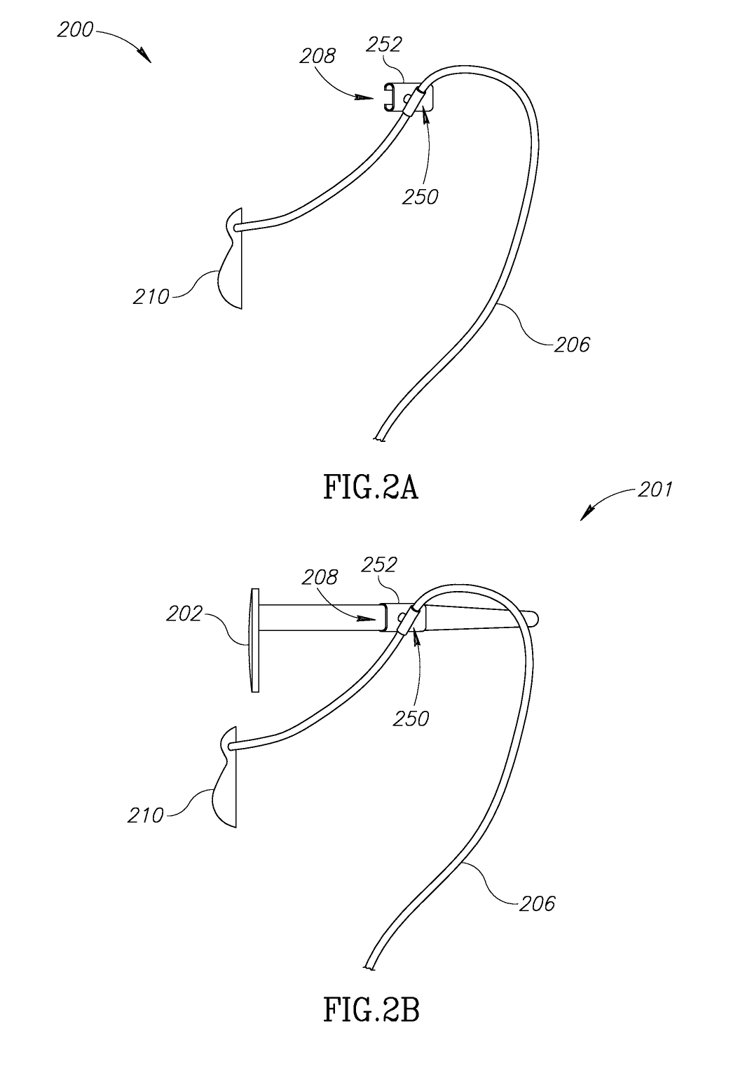

Reference is now made to FIG. 2A, which schematically illustrates a breath sampling device 200 with a connector 208, according to some embodiments. According to some embodiments, connector 208 comprises a support structure connector 252 configured to connect to a support structure, and a sample tubing connector 250 configured to connect to a tube. As illustrated in FIG. 2A, tubing connector 250 is connected to a tubing system 206 that is configured to convey breath samples obtained by a breath sample collector 210 connected thereto. Breath sample collector 210 is configured to obtain respiratory gas samples from a respiratory opening of a user (not shown) and deliver the samples to tubing system 206.

According to some embodiments, the samples may then be delivered form tubing system 206 to a measuring device (not shown) for evaluating a breath related parameter of index.

Reference is now made to FIG. 2B, which schematically illustrates a breath sampling device 201 with a connector 208 and an eyewear support structure 202, according to some embodiments. According to some embodiments, connector 208 comprises a support structure connector 252 configured to connect to a support structure, and a sample tubing connector 250 configured to connect to a tube. As illustrated in FIG. 2B, tubing connector 250 is connected to a tubing system 206 that is configured to convey breath samples obtained by a breath sample collector 210 connected thereto. Breath sample collector 210 is configured to obtain respiratory gas samples from a respiratory opening of a user (not shown) and deliver the samples to tubing system 206, and support structure connector 252 is connected to eyewear support structure 202. Eyewear support structure 202 is configured to be worn by a subject and provide structural support for breath sampling device 201.

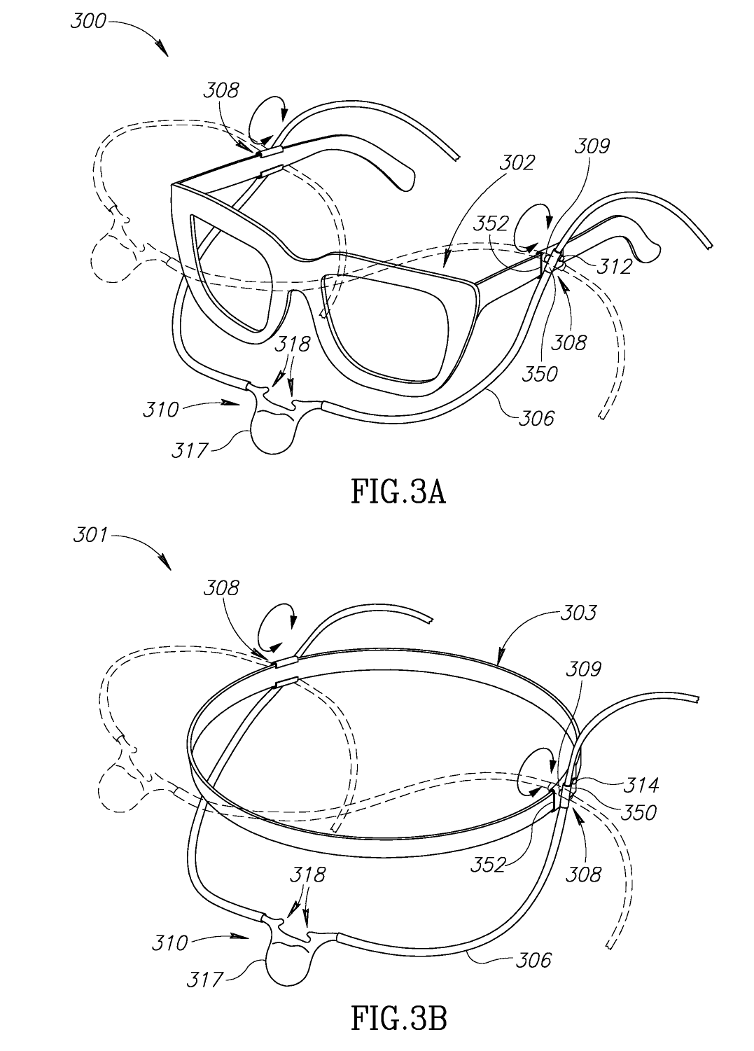

Reference is now made to FIG. 3A, which schematically illustrates a breath sampling device 300 with a connector 308, a positioning element 309 and an eyewear support structure 302 in a perspective view, according to some embodiments. According to some embodiments, connector 308 includes a support structure connector 352 connected to eyewear support structure 302, and a sample tubing connector 350 connected to a tubing system 306. Tubing system 306 is configured to convey breath samples obtained by a breath sample collector 310 connected thereto, to be analyzed for evaluating respiratory related parameters. Breath sample collector 310 contains two nasal sample collectors 318 for collecting respiratory gas samples from within or near the nostrils of a user, and an oral sample collector 317 for collecting respiratory gas samples from the mouth of the user. Breath sample collector 310 is configured to deliver the samples obtained by nasal sample collectors 318 and oral sample collector 317 to tubing system 306.

Connector 308 further includes positioning element 309 placed between sample tubing connector 350 and support structure connector 352. Positioning element 309 is configured to facilitate changes in the position of sample tubing connector 350 in relation to (respective to) support structure connector 352, thereby changing the position of tubing system 306 and sample collector 310 in relation to (respective to) eyewear support structure 302.

FIG. 3A, illustrates tubing system 306 and sample collector 310 in a first position (continuous line) at which sample collector 310 is approximated to a respiratory opening of a subject (not shown) for obtaining respiratory gas samples, and in a second position (dotted line) at which sample collector 310 is retracted from a respiratory opening of a subject (not shown) to facilitate accessibility to the respiratory opening.

According to some embodiments, breath sampling device 300 further includes an indication mechanism, such as a micro-switch 312, configured to provide indication regarding the position of positioning element 309 or the relative position between tubing system 306 and eyewear support structure 302. According to some embodiments, the indication mechanism is automatically actuated upon changes in positions of positioning element 309. According to some embodiments, the indication mechanism is manually actuated, for example by a user. Micro-switch 312 is placed on connector 308 and is automatically actuated by position changes of sample tubing connector 350 which presses against it in the second position, and releases the pressing in the first position.

According to some embodiments, the indications from micro-switch 312 are delivered to an analysis unit (not shown) which may modulate the sampling activity accordingly.

According to some embodiments, tubing system 306 is integrally formed with sample tubing connector 350. According to some embodiments, support structure connector 352 is integrally formed with eyewear support structure 302. According to some embodiments support structure connector 352 is removably/reversibly/detachably connected with eyewear support structure 302. According to some embodiments, sample tubing connector 350 is removably/reversibly/detachably connected with tubing connector 350.

Reference is now made to FIG. 3B, which schematically illustrates a breath sampling device 301 with a connector 308, a positioning element 309 and a headband support structure 303 in a perspective view, according to some embodiments. According to some embodiments, connector 308 includes a support structure connector 352 connected to headband support structure 303, and a sample tubing connector 350 connected to a tubing system 306. Tubing system 306 is configured to convey breath samples obtained by a breath sample collector 310 connected thereto, to be analyzed for evaluating respiratory related parameters. Breath sample collector 310 contains two nasal sample collectors 318 for collecting respiratory gas samples from within or near the nostrils of a user, and an oral sample collector 317 for collecting respiratory gas samples from the mouth of the user. Breath sample collector 310 is configured to deliver the samples obtained by nasal sample collectors 318 and oral sample collector 317 to tubing system 306.

Connector 308 further includes positioning element 309 placed between sample tubing connector 350 and support structure connector 352. Positioning element 309 is configured to facilitate changes in the position of sample tubing connector 350 in relation to (respective to) support structure connector 352, thereby changing the position of tubing system 306 and sample collector 310 in relation to (respective to) headband support structure 303.

FIG. 3B, illustrates tubing system 306 and sample collector 310 in a first position (continuous line) at which sample collector 310 is approximated to a respiratory opening of a subject (not shown) for obtaining respiratory gas samples, and in a second position (dotted line) at which sample collector 310 is retracted from a respiratory opening of a subject (not shown) to facilitate accessibility to the respiratory opening.

According to some embodiments, breath sampling device 300 further includes an indication mechanism, such as a micro-switch 314, configured to provide indication regarding the position of positioning element 309 or the relative position between tubing system 306 and headband support structure 303. According to some embodiments, the indication mechanism is automatically actuated upon changes in positions of positioning element 309. According to some embodiments, the indication mechanism is manually actuated, for example by a user. Micro-switch 314 is placed on connector 308 and is automatically actuated by position changes of sample tubing connector 350 which presses against it in the second position, and releases the pressing in the first position.

According to some embodiments, the indications from micro-switch 314 are delivered to an analysis unit (not shown) which may modulate the sampling activity accordingly.

According to some embodiments, tubing system 306 is integrally formed with sample tubing connector 350. According to some embodiments, support structure connector 352 is integrally formed with headband support structure 303. According to some embodiments support structure connector 352 is removably/reversibly/detachably connected with headband support structure 303. According to some embodiments, sample tubing connector 350 is removably/reversibly/detachably connected with tubing connector 350.

According to some embodiments, connector 308 includes a lock clip. According to some embodiments, connector 308 includes a catch clip. According to some embodiments, connector 308 includes a hasp clip. According to some embodiments, connector 308 is a clip.

According to some embodiments, tubing system 306 comprises one or more tubes that may be the same or different from each other.

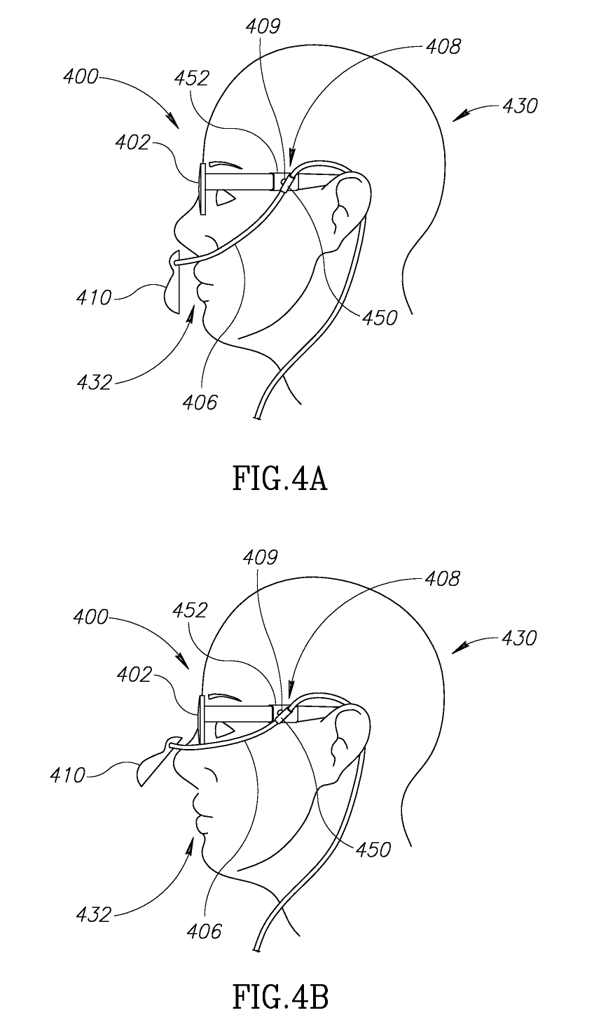

Reference is now made to FIG. 4A and 4B which schematically illustrate a breath sampling device 400 with an eyeglass frame 402 on a subject 430, according to some embodiments. According to some embodiments, breath sampling device 400 includes a sample collector 410 designed to collect breath samples from a mouth 432 of subject 430. Sample collector 410 is connected to tubing system 406 which is configured to deliver the samples collected by sample collector 410 to an analysis unit (not shown) for measuring parameters related to the respiration of subject 430. Tubing system 406 is connected to eyeglass frame 402 via a connector 408. According to some embodiments, connector 408 includes a support structure connector 452 connected to eyeglass frame 402, and a sample tubing connector 450 connected to a tubing system 406. According to some embodiments, connector 408 further includes a positioning element 409 placed between sample tubing connector 450 and support structure connector 452. Positioning element 409 is configured to facilitate changes in the position of sample tubing connector 450 in relation to(respective to) support structure connector 452, thereby changing the position of tubing system 406 and sample collector 410 in relation to (respective to) eyeglass frame 402.

In FIG. 4A sample collector 410 and tubing system 406 are positioned in a first “engaged” position. At this position, sample collector 410 is configured to collect respiratory samples from mouth 432 of subject 430. Access to mouth 432 is at least partially impaired/obstructed by sample collector 410 in this position.

In FIG. 4B sample collector 410 and tubing system 406 are positioned in a second “partially-retracted” position. At this position, sample collector 410 is rotated upwards away from mouth 432 and access to mouth 432 is gained/facilitated.

According to some embodiments, there is provided a tubing system (sampling line) configured to be attached via clips to a frame of eyeglasses. According to some embodiments, the tubing system is integrally formed with a frame of eyeglasses. According to some embodiments, the frame includes a frame extension configured to support the tubing system. According to some embodiments, the tubing system is pliable. According to some embodiments, the tubing system is attached to a pliable element such as a stiff and flexible rod, the rod is pliable to obtain a desired shape and retain the desired shape. According to some embodiments, the desired shape and support frame are designed to facilitate breath sample collection/gathering from a respiratory opening of the subject without having the tubing system and/or the sample collector touching the nose or mouth of the subject. According to some embodiments, the tubing system and/or the pliable rod is configured to be shaped according to the exact geometry of each subject.

According to some embodiments, a pliable element may include a pliable rod configured to be attached to a portion of the sampling tubing system. According to some embodiments, the pliable element is stiff after positioning.

According to some embodiments, a pliable element may include a pliable conduit and/or pipe configured to be attached to a portion of the sampling tubing system. According to some embodiments, the pliable element may include molded plastic, stiff metal wire, or formed metal.

According to some embodiments, a pliable element may be integrally formed with the sampling tubing system.

According to some embodiments, the tubing system is configured to be connected to a flip mechanism on the frame of the eyeglasses. The flip mechanism is configured to facilitate positioning the tubing system at different positions. According to some embodiments, a switch is introduced to indicate to an analysis-unit or a processing circuitry the position of the tubing system.

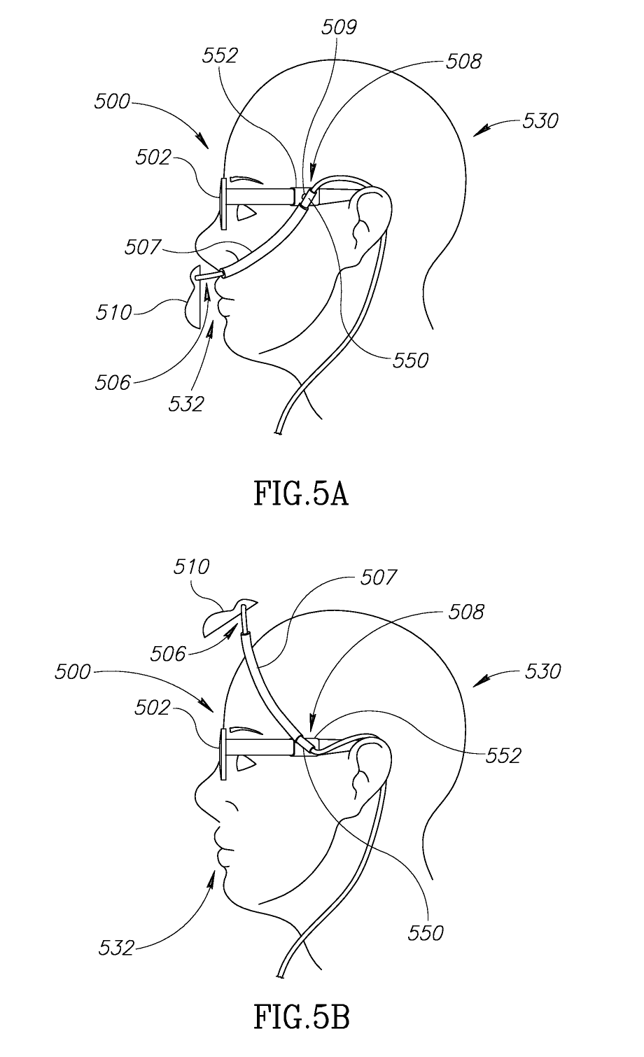

Reference is now made to FIG. 5A and 5B which schematically illustrate a breath sampling device 500 with an eyeglass frame 502 and a pliable conduit 507 on a subject 530 according to some embodiments. According to some embodiments, breath sampling device 500 includes a sample collector 510 designed to collect breath samples from a mouth 532 of subject 530. Sample collector 510 is connected to a tubing system 506 which is configured to deliver the samples collected by sample collector 510 to an analysis unit for measuring parameters related to the respiration of subject 530. Tubing system 506 is connected to eyeglass frame 502 via a connector 508. Connector 508 is configured to facilitate moving tubing system 506 and sample collector 510 between a first position and a second position.

According to some embodiments, a pliable structure, such as pliable conduit 507, is at least partially attached to tubing system 506 and configured to be bent and to retain the bent shapes, thereby holding tubing system 506 or parts thereof at desired shapes.

According to some embodiments, tubing system 506 and pliable conduit 507 are pliable to obtain a desired position at which sample collector 510 and tubing system 506 do not come in direct contact with a face of subject 530.

According to some embodiments, connector 508 includes a support structure connector 552 connected to eyeglass frame 502, and a sample tubing connector 550 connected to a tubing system 506. According to some embodiments, connector 508 further includes a positioning element 509 placed between sample tubing connector 550 and support structure connector 552. Positioning element 509 is configured to facilitate changes in the position of sample tubing connector 550 in relation to (respective to) support structure connector 552, thereby changing the position of tubing system 506 and sample collector 510 in relation to (respective to) eyeglass frame 502.

In FIG. 5A sample collector 510 and tubing system 506 are positioned in a first “engaged” position. At this position, sample collector 510 is configured to collect respiratory samples from mouth 532 of subject 530. Access to mouth 532 is at least partially impaired/obstructed by sample collector 510 in this position.

In FIG. 5B sample collector 510 and tubing system 506 are positioned in a second “retracted” position. At this position, sample collector 510 is rotated upwards away from mouth 532 and access to mouth 532 is gained/facilitated.

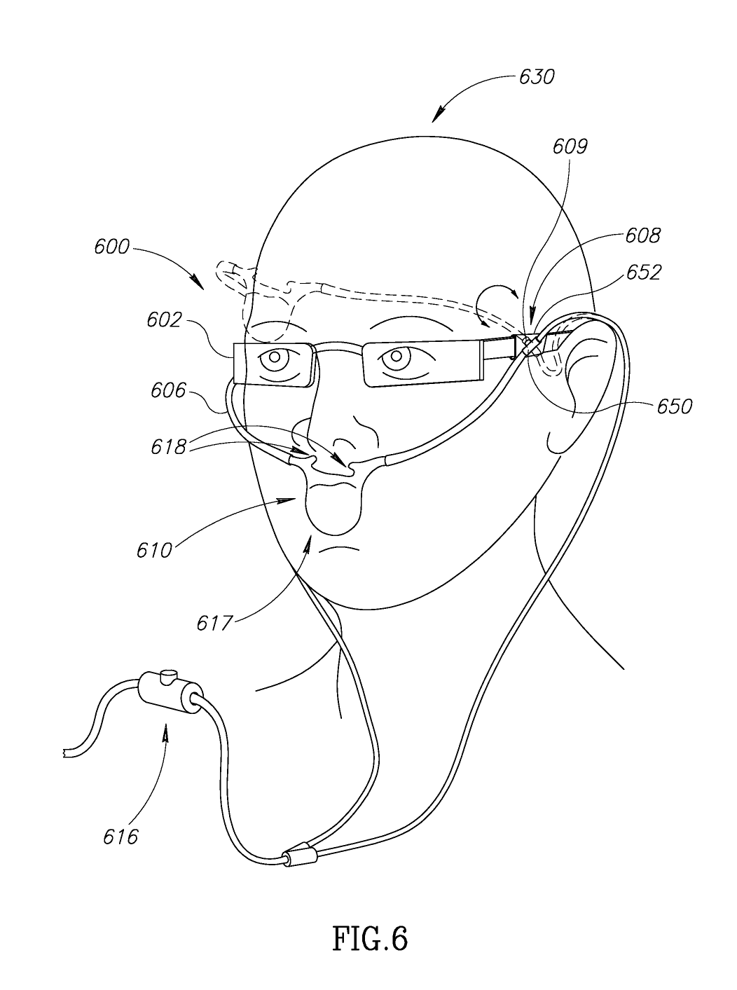

Reference is now made to FIG. 6 which schematically illustrates a breath sampling device 600 with an eyeglass frame 602 and a manual switch 616 on a subject 630, according to some embodiments. According to some embodiments, breath sampling device 600 includes a breath sample collector 610 designed to collect breath samples from a mouth of subject 630.

According to some embodiments, breath sample collector 610 contains two nasal sample collectors 618 for collecting respiratory gas samples from within or near the nostrils of a user, and an oral sample collector 617 for collecting respiratory gas samples from the mouth of the user. Breath sample collector 610 is configured to deliver the samples obtained by nasal sample collectors 618 and oral sample collector 617 to tubing system 606, which is configured to deliver the samples collected by sample collector 610 to an analysis unit for measuring parameters related to the respiration of subject 630. Tubing system 606 is connected to eyeglass frame 602 via connector 608.

According to some embodiments, connector 608 includes a support structure connector 652 connected to eyeglass frame 602, and a sample tubing connector 650 connected to a tubing system 606. According to some embodiments, connector 608 further includes a positioning element 609 placed between sample tubing connector 650 and support structure connector 652. Positioning element 609 is configured to facilitate changes in the position of sample tubing connector 650 in relation to (respective to) support structure connector 652, thereby changing the position of tubing system 606 and sample collector 610 in relation to (respective to) eyeglass frame 602.

Connector 608 is configured to facilitate moving tubing system 606 and sample collector 610 between a first “engaged” position (continuous line) and a second “retracted” position (dotted line). Additionally, breath sampling device 600 includes a manually actuated indication element, such as manual switch 616, which is configured to allow subject 630 to provide indication to an analysis unit related to the position of tubing system 606 and sample collector 610. Switch 616 is manually actuated by subject 630 or a medical care giver thereof. For example, when subject 630 or a professional care giver positions tubing system 606 and sample collector 610 in the second position, they press switch 616 to indicate to a detector or an analyzing unit that tubing system 606 and sample collector 610 are retracted from a mouth and/or a nose of patient 630.

According to some embodiments, an analyzing unit and/or detector may obtain the signal indicative of the position of a sample collector. When the signal indicates that the sample collector is in a retracted position, the analyzing unit and/or detector may temporarily stop the detection and/or analysis operation until the signal indicates that the sample collector is returned to an engaged position.

According to some embodiments, the button is manually controlled by a subject or a care giver. According to some embodiments, the button is automatically controlled upon detecting changes in the position of the sample collector and/or the tubing system.

According to some embodiments, the cannula or parts thereof comprise a flexible elastic tubing system attached to bendable rods. The bendable rods are configured to hold the tubing system in a desired position.

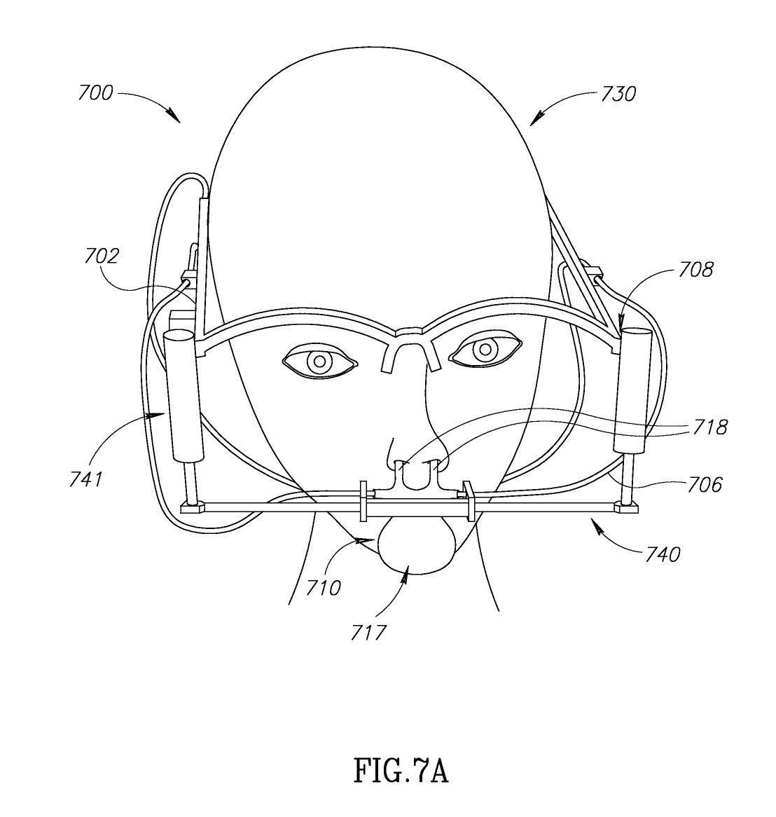

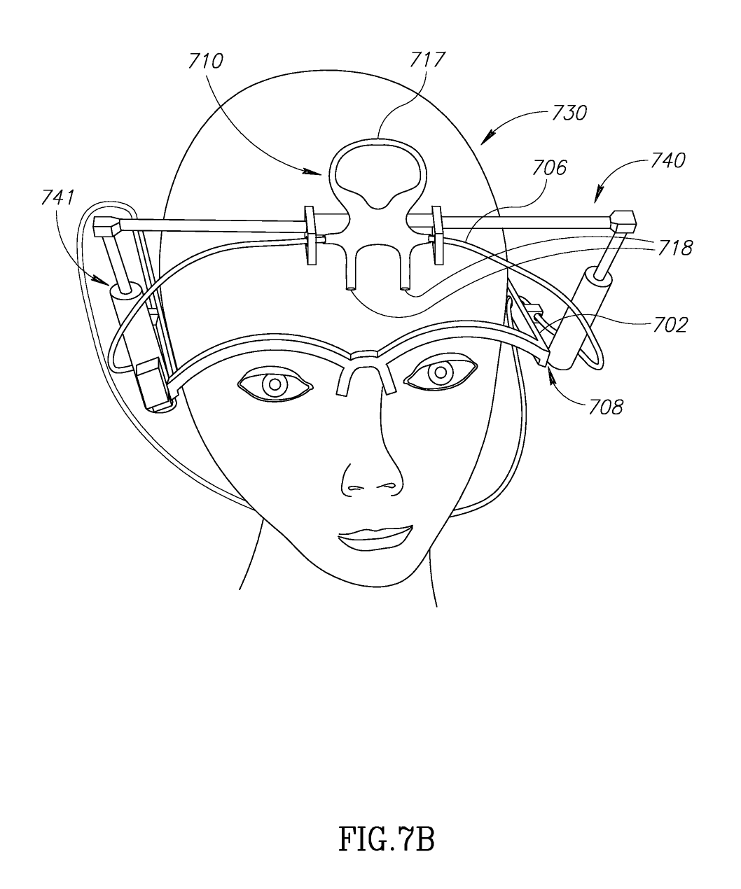

Reference is now made to FIG. 7A and FIG. 7B, which schematically illustrate a breath sampling device 700 with an eyewear support structure, such as eyeglass frame 702, and a tube holder 740 on a subject 730 in an engaged position and a retracted position respectively, according to some embodiments. According to some embodiments, breath sampling device 700 includes a breath sample collector 710 designed to collect breath samples from subject 730 using two nasal sample collectors 718 for collecting respiratory gas samples from within or near the nostrils of a user, and an oral sample collector 717 for collecting respiratory gas samples from the mouth of the user. Breath sample collector 710 is configured to deliver the samples obtained by nasal sample collectors 718 and oral sample collector 717 to a tubing system 706, which is configured to deliver the samples collected by sample collector 710 to an analysis unit (not shown) for measuring parameters related to the respiration of subject 730. Tubing system 706 is connected to tube holder 740 which holds tubing system 706 at desired positions. According to some embodiments, tube holder 740 is mechanically connected to eyeglass frame 702 through connector(s) 708 which is configured to permit the altering of the position of tube holder 740 between different positions.

According to some embodiments, tube holder 740 may include movable/bendable parts/portions to allow for adaptation and positioning of sample collector 710 according to the geometries of patient 730.

According to some embodiments, tube holder 740 may be adjustable to fit the respiratory openings of subject 730. According to some embodiments, tube holder 740 may include a positioning piston 741 for adjusting tube holder 740 to fit the respiratory openings of subject 730. According to some embodiments, tube holder 740 may include displaceable elements/parts. According to some embodiments, tube holder 740 may have a “U” shape. According to some embodiments, tube holder 740 may have a “C” shape.

In FIG. 7A sample collector 710 and tubing system 706 (and tube holder 740) are positioned in a first “engaged” position. At this position, sample collector 710 is configured to collect respiratory samples from the face of patient 730. This position facilitates collecting samples from the respiratory gasses of patient 730.

In FIG. 7B sample collector 710 and tubing system 706 (and tube holder 740) are positioned in a second “retracted” position. At this position, sample collector 710 is rotated upwards away from the face of patient 730 and access to the mouth or nose of patient 730 is facilitated.

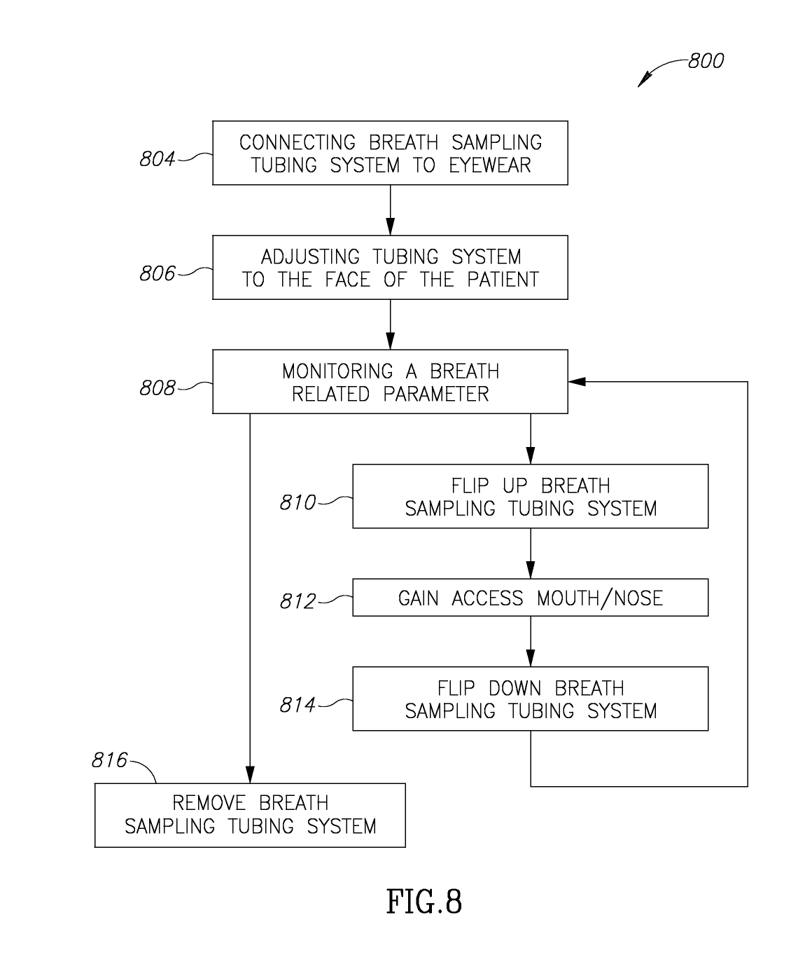

Reference is now made to FIG. 8 which illustrates a block diagram 800 of a method for using a breath sampling device with a displaceable sample collector, according to some embodiments. The method includes connecting a breath sampling tubing system to an eyewear (step 804), adjusting the breath sampling tubing system to the face of the subject (step 806), and monitoring a breath related parameter (step 808). Then, when access to a mouth and/or nostril of the subject is required, the method includes flipping up breath sampling tubing system (step 810), to gain access to a mouth/nose of the subject (step 812) and, when the access is no longer needed, flipping down breath sampling tubing system (step 814) to resume the monitoring a breath related parameter (step 808), until the monitoring is finished and the monitoring a breath related parameter (step 808) is removed (step 816).

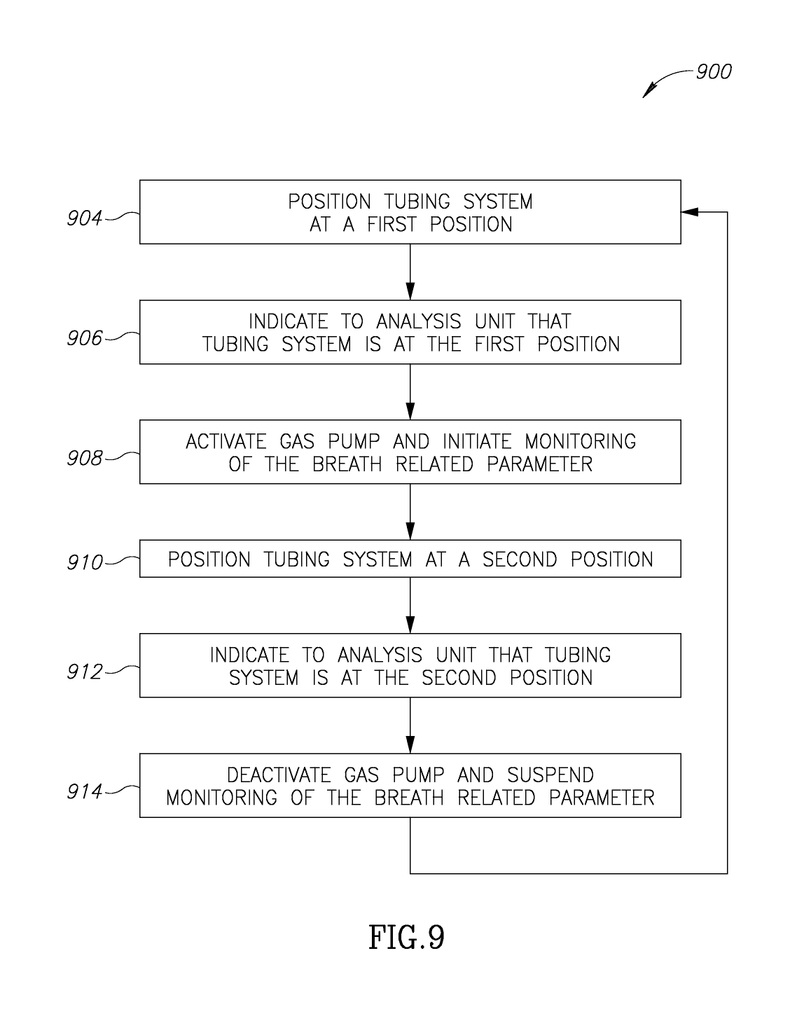

Reference is now made to FIG. 9, which illustrates a block diagram 900 of a method for using a breath sampling device with a displaceable sample collector and an indication mechanism, according to some embodiments. According to some embodiments, the method includes positioning the tubing system at a first “engaged” position (step 904), indicating to an analysis unit that the tubing system is at a first position (step 906), activating a gas pump and initiating monitoring of a breath related parameter (step 908), positioning tubing system at a second “retracted” position (step 910), indicating to an analysis unit that tubing system is at a second position (step 912), deactivating gas pump and suspending the monitoring of the breath related parameter (step 914).

According to some embodiments, if monitoring of the breath related parameter is still required, the method includes, again, positioning the tubing system at a first “engaged” position (step 904) and following the succeeding steps as mentioned above.

According to some embodiments, the term “wearable support structure” may refer to any device or structure configured to be wearable by a subject, for example, on a subject's head, face, neck, ear(s), nose or the like, or any combination thereof, and provides support to a connector, which is a part of a breath sampling apparatus.

According to some embodiments, the wearable support structure may include an eyeglass frame, a head band, earphones, headset, hearing aid, hair pin, hair accessory or the like, or any combination thereof.

According to some embodiments, the term “connector” may refer to any device or structure configured to connect a breath sampling tube to a wearable support structure. The connector may include a support structure component for connecting to the support structure. The support structure component may include a sleeve, a clip, a clamp, a magnet, a spring, a stent, a tube, an adhesive member, Velcro™, or the like, or any combination thereof, for connecting to the wearable support structure.

The connector may further include a tube component for connecting to the breath sampling tube. The tube component may include a sleeve, a clip, a clamp, a magnet, a spring, a stent, a tube, an adhesive member, Velcro™, or the like, or any combination thereof, for connecting to the breath sampling tube.

According to some embodiments, the connector may further include a positioning element configured to move the support structure component and the tube component of the connector relative to one another. The positioning element is thus movable, for example, between a first position and a second position such that at the first position said oral/nasal breath sample collector, which is connected to the breath sampling tube, is approximated to the respiratory opening of the subject for collecting breath samples, and at the second position said oral/nasal breath sample collector is retracted from the respiratory opening of the subject to facilitate accessibility to the respiratory opening.

According to some embodiments, the “positioning element” may include any device or structure such as a screw, pin, bolt, joint, magnet(s), disk(s), gear(s), plastic structure with detents, slides, or the like, or any combination thereof.

According to some embodiments, the connector is movable between a first position and a second position such that at the first position said oral/nasal breath sample collector is approximated to the respiratory opening of the subject for collecting breath samples, and at the second position said sample collector is retracted from the respiratory opening of the subject to facilitate accessibility to the respiratory opening.

According to some embodiments, the term “indication mechanism” may refer to any device, structure, electric component, electromechanical component (such as a switch, piezoelectric structure, and others), configured to provide a signal to an analysis unit (such as a gas/breath analyzer, such as a capnograph) and or a pump, wherein the signal is indicative of the position of the connector, positioning element, breath sampling tube and/or the oral/nasal breath sample collector.

According to some embodiments, the term “approximated” or “approximated position” may refer to a position in which the sample collector is in close proximity to a respiratory opening of a subject to facilitate collecting respiratory gas samples. According to some embodiments, the term “approximated” or “approximated position” may refer to a position in which the sample collector is in direct contact with a respiratory opening of a subject to facilitate collecting respiratory gas samples.

According to some embodiments, the term “approximated” or “approximated position” may refer to a position in which the sample collector and/or the sampling tubing system are near, and do not come indirect contact with the face of a subject.

According to some embodiments, the term “pliable” may refer to the characteristics of a material being stiff and flexible. According to some embodiments, the term “pliable” may refer to the characteristics of a material being bendable to certain shapes, and able to retain the bent shape after the bending process.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A breath sampling apparatus, comprising:
a breath sampling tube;
a connector connectable to or formed with said breath sampling tube configured to attach said breath sampling tube to a wearable support structure; and
an oral/nasal breath sample collector configured to collect breath samples from a patient's respiratory opening and deliver the collected breath samples to said breath sampling tube;
wherein said connector comprises a positioning element movable between a first position and a second position such that at the first position said oral/nasal breath sample collector is approximated to the respiratory opening of the subject for collecting breath samples, and at the second position said oral/nasal breath sample collector is retracted from the respiratory opening of the subject to facilitate accessibility to the respiratory opening.

2. The apparatus of claim 1, wherein said wearable support structure comprises eyeglasses.

3. The apparatus of claim 1, wherein said wearable support structure comprises a head band, earphones, a headset, a hearing aid, a hair pin or a hair accessory.

4. The apparatus of claim 1, wherein said oral/nasal breath sample collector is configured to obtain respiratory samples from a mouth and/or one or two nostrils of the subject.

5. The apparatus of claim 1, wherein said oral/nasal breath sample collector further comprises one or more oxygen delivery outlets.

6. The apparatus of claim 1, further comprising an indication mechanism configured to provide indication related to a position of said breath sample collector to a breath analyzer, wherein the breath analyzer is configured to modulate breath sampling and/or breath analyzing upon indication that said positioning element is at the second position.

7. The apparatus of claim 1, further comprising an indication mechanism configured to provide indication related to a position of said oral/nasal breath sample collector to a breath analyzer, wherein the breath analyzer is configured to activate breath sampling and/or breath analyzing upon indication that said positioning element is at the first position.

8. The apparatus of claim 1, further comprising an indication mechanism configured to provide indication related to a position of said breath sample collector to a pump, wherein the pump is configured to stop breath sampling upon indication that said positioning element is at the second position.

9. The apparatus of claim 1, further comprising an indication mechanism configured to provide indication related to a position of said breath sample collector to a pump, wherein the pump is configured to activate breath sampling upon indication that said positioning element is at the first position.

10. The apparatus of claim 6, wherein said indication mechanism is configured for manual actuation.

11. The apparatus of claim 6, wherein said indication mechanism is configured for automatic actuation upon changes in a position of said positioning element.

12. The apparatus of claim 1, wherein said breath sampling tube is pliable and configured to retain desired shapes.

13. The apparatus of claim 1, wherein said breath sampling tube comprises a pliable element configured to retain desired shapes.

14. A wearable support device for breath sampling, the device comprising:
a support structure, wearable by a subject; and
a connector connectable to or formed with said support structure and configured to attach said support structure to a breath sampling tube,
wherein said connector comprises a positioning element movable between a first position and a second position such that at the first position said breath sampling tube is approximated to the respiratory opening of the subject for collecting breath samples, and at the second position said breath sampling tube is retracted from the respiratory opening of the subject to facilitate accessibility to the respiratory opening.

15. The device of claim 14, further comprising an indication mechanism configured to provide indication related to a position of said breath sampling tube to a breath analyzer, wherein the breath analyzer is configured to modulate breath sampling and/or breath analyzing upon indication that said positioning element is at the second position.

16. The device of claim 15, wherein said indication mechanism is configured for manual actuation.

17. The device of claim 15, wherein said indication mechanism is configured for automatic actuation upon changes in a position of said positioning element.

18. A method for breath sampling from a subject, the method comprising:
connecting a breath sampling tube having an oral/nasal breath sample collector to a wearable support structure, such that the breath sampling tube is movable relative to the wearable support structure;
using an indication mechanism, providing a signal indicative of a position of said oral/nasal breath sample collector to a breath analyzer and/or a pump;
using processing circuitry, modulating the activity of the breath analyzer and/or the pump based on an indication received from the indication mechanism.

19. The method of claim 18, wherein the breath sampling tube is flexible and configured to retain desired shapes by bending.

20. The method of claim 19, further comprising adjusting the breath sampling tube to the subject by bending it.
